SEQUENCE LISTING (15 sequences)

SEQ ID NO: 1 - protein, 411 aa, Cuphea palustris
  acyl-ACP thioesterase
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSNGNSIS

SEQ ID NO: 2 - protein, 329 aa, Argyrotaenia velutinana
  acyl-CoA desaturase
MAPNAEDIETNMPETEENWETLVAPQAAPRKYQIVYKSLLTFGYGHLAGLYGLYLCFTSAKWQTIGLAIILHAMAILGITAGAHRLWTHRAYKATVPLQIILIIFNSLSFQNSAFTWIRDHRLHHKYSDTDGDPHNATRGFFYSHIGWLLVRKHPEVMKRGRMTEMSDIYSNPIIMFQKNYAIPFIGTVCFVLPTIIPMYFWGETLNNAWHITVLRYIFSLNCIFLVNSAAHLYGYKPYDKNILPAENKAASIASFGEAFHNYHHVFPWDYRASELGNITMNWTIYFIDFFAWIGWAYDLKTASDETIKSRIKRTGDGTDFSGQQIYAR

SEQ ID NO: 3 - protein, 335 aa, Choristoneura rosaceana
  acyl-CoA desaturase
MAPNVEDMESDLPESEEKLEKLVAPQAAPRKYQIIYTNLLTFGYWHIAGLYGLYLCFTSAKWQTIILALILNEMAILGITAGAHRLWAHRSYKATVPLQIILIIFNSLSFQNSAIHWIRDHRMHHKYSDTDGDPHNASRGFFYSHVGWLLVKKHPEVKKRAKTIDMSDIYSNPILRFQKKYAIPFIGMICFVLPTIIPMYFWGETLSNAWHITMLRYVFSLNSIFLVNSAAHLYGYRPYDKNILPAENKMTFIACLGENFHNYHHVFPWDYRASELGNIGMNWTAKFIDFFAWIGWAYDLKTASDENIKSRMKRTGDGTDVSGQKYSCESSEVLQ

SEQ ID NO: 4 - protein, 334 aa, Choristoneura parallela
  acyl-CoA desaturase
MAPNVEDMESDMPESEKWEKLVAPQAAPRKYEIIYTNLLTFGYGHIAGLYGLYLCFTSAKWQTVILAIILNEMAILGITAGAHRLWSHRSYKAAVPLQIILMIFNSLAFQNSAINWVRDHRMHHKYSDTDGDPHNASRGFFYSHVGWLLVKKHPEVKKRGKMIDMSDIYSNPVLRFQKKYAIPFIGMICFVLPTIIPMYFWGETLSNAWHITMLRYVFSLNSIFLVNSAAHLYGYRPYDKNILPAENKIALIACLGDSFHNYHHVFPWDYRASELGNIGMNWTAQFIDFFAWIGWAYDLKTASDENINSRMKRTGDGTDISGQKYSCESSEVLQ

SEQ ID NO: 5 - protein, 329 aa, Ostrinia nubilalis
  acyl-CoA desaturase
MVPYATTADGHPEKDECFEDNEIKSNSLPKLEILYFNVMTFTFLHLSALYGLYLGFTSVKWATIGLGIIFYFFAEIGITAGAHRLWSHRSYKAKLPLEILLMVFNSMAFQNTALSWARDHRVHHKCPDTNGDPHNANRGFFYSHVGWLMTKKSDEVIKQGKLCDVADLYSNPVLRFQKKYAVPFIGTLCFVLPTLIPMYFWGETLNNAWHFNMFRYVINLNATFCVNSVVHKWGYKPYDKNICPTQNVLLNLAVLGEAFHNYHHVFPWDYRAAELGNQKMNPTTLFIDFFAWIGWAYDLKTASKEMIKSRSERTGDGTDLWGHSADKLK

SEQ ID NO: 6 - protein, 326 aa, Amyelois transitella
  acyl-CoA desaturase
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 7 - protein, 455 aa, Helicoverpa armigera
  fatty acyl reductase
MVVLTSKETKPSVAEFYAGKSVFITGGTGFLGKVFIEKLLYSCPDIENIYMLIREKKGLSVSERIKQFLDDPLFTRLKDKRPADLEKIVLIPGDITAPDLGINSENEKMLIEKVSVIIHSAATVKFNEPLPTAWKINVEGTRMMLALSRRMKRIEVFIHISTAYTNTNREVVDEILYPAPADIDQVHQYVKDGISEEDTEKILNGRPNTYTFTKALTEHLVAENQAYVPTIIVRPSVVAAIKDEPLKGWLGNWFGATGLTVFTAKGLNRVIYGHSSYIVDLIPVDYVANLVIAAGAKSSKSTELKVYNCCSSSCNPVTIGTLMSMFADDAIKQKSYAMPLPGWYIFTKYKWLVLLLTFLFQVIPAYVTDLSRHLIGKSPRYIKLQSLVNQTRSSIDFFTNHSWVMKADRVRELYASLSPADKYLFPCDPTDINWTHYIQDYCWGVRHFLEKKSYE

SEQ ID NO: 8 - protein, 462 aa, Ostrinia nubilalis
  fatty acyl reductase
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHVAAKLLFKMSLTAAVNINTKPTEQLIAICKKMRRNPIFIYVSSAYSNVNEQIIDEKVYNTGVPLETIYDTLDTENTRITDIFLDKRPNTYTYSKALAEVVVEKEFDESAAIVRPSIIVSSIREPIPGWLSGSHGFPRVVGAACKGLLLRWHGDGTVVCDLIPVDHVANLIIAAAWESNERRLMGNKGVKVYNCCSSLRNPIDVITVVKTCIKYRKYFGTRTMSIFTPRFIMKKNYFIYKLLYFTYHTIPAAIIDGFFWLTGRTPIMLKTLDKLGKISSVLEYFTHHQFIFLDSNVRGLLRRMEGTDRQTFNFDVTEIEWEPYLQNFVRGIANNYDYSM

SEQ ID NO: 9 - protein, 459 aa, Ostrinia nubilalis
  fatty acyl reductase
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHLAAKLLFKMSLAAAVNINTKSTEQLIAICKKMRRNPIFIYVSSAYSNVNKQIIDEKVYSTGVPLETIYDTLDAKNTRLMDIFLDKRPNTYTYSKALAEVLVENEFDESAAIVRPSIIASSIREPIPGWLSGSHGFPRVVEAACKGLLLRWHGDGTVAFGIIPVDHVANLIIAAAWESNERRLIGNKGVKVYNCCSGLRNPIDVSTVMNTCLKYRKYFGTRTMSIITPRFIMKKNYFLYKLLYFTYHTIPAAIIDGFFWLTGRTPMMLNTLHKLRKLSSVLEYFTLRQFLFLDSNVRGLLRRMEGTDRQTFNFDVTEIEWEPFLQNCVRGIANNYD

SEQ ID NO: 10 - protein, 449 aa, Yponomeuta rorrellus
  fatty acyl reductase
MVQLKEDSVAAFYAEKSIFITGGTGFLGKVLIEKLLYSCKAVDQIYVLIRKKKDQTPSERIAQLLESELFSRLRKDDPSALKKVVPVVGDLTMPNLGLSAAVEDLIVSKVTVIFHVAATVKFNERMKNALVNNVEATREVINLCHRLEKVDAFIHVSTAYSNTDQKVVEERVYPPPAPLSEVYAFVKNYGDDMDIIQNLLNGRPNTYTYTKALAEDIVLKEHGGIPTAIIRPSIVLSVLKEPIPGWLDNWNGPTGLLHASSQGVHCSMLGSGSNVADLIPVDIVTNLMIVVASRCRKSNGLKVYNSCSGTTNPITYQAFTKMFLDSCISRGWNKVPFPLLIFVKWAFLNRVLKFLLVIVPFFLIDVYLRFFGKPNYMRMITYTKKAEDLMTFFTSHEWQFKDGNVRDLINMMSPEDRKIFYCDPEEIQWKPYFDDYCVGVFKYLLKRKV

SEQ ID NO: 11 - protein, 368 aa, Euglena gracilis
  wax synthase
MDFLGFPDSESERHAHFYVLASSFAAAIYMFTIPRRVKAGRKRFLLCSPVLLLNIMQPYIFFWTVGRHYCNFIPLYAAFCTWWTAFKVMAFGIGRGPLCQFSAFHKFAVVMLLPILPHGDTNHGVKDERSGSSWSSPTYLEMFAKFCGLGLCTYGISQLSHDGFPVLYNVFLSLIMYLHICVQYTGSNLATSKVLQVPLSDGMNQPYFSTSLSNFWGRRWNLVASSSLRHVVYDPIREGRLVPKGHPEEKPGGGKEVSRKVLGSLMAFLVSGIMHEYILWLATGFWSGQMLLFFVVHGVAVAAERVAKVAWARHGLPAIPCAVSIPMTIGFLFGTAELLFYPPIFSANWAEHGVADLRRQFRSLGLSV

SEQ ID NO: 12 - protein, 290 aa, Gallus gallus
  wax synthase
MKIEFAPLSLPLQRRLQTAAVVQWVFSFLCLAQCCTAAFIGLLFTRFWLLSVLYAAWWFVDREAPLRGGRRIHMVRNSAVWRHMRDFFPVTLVKTAELDPRQNYLVGFHPHGVLAVGAFINFGTEATGFSTIFPGITPHLMMLSLWFRVPFLRDYLMSGGLVSSDKESAYHVLQRPEGGNLLAIIVGGAQEALDARPGSCTLLLKNRKGFVRVAIEQGTPLVPAFSFGENELFDQVSNPKGSWLRWIQHRLQQIMGISLPLFHARGIFQYSFGLVPYRRPINTLGSQFPC

SEQ ID NO: 13 - protein, 362 aa, Gallus gallus
  wax synthase
MKTIIAACSQNLSGSRASLHAALRTLLAVPWPSQRDVRAWLQLLAVLQWVLSFLLLGPVTLVLLIYLVFTRFWPISALYLAWVIFDWDTPEKGGRRLPCLRRWSVWRHFRDYFPVKLVKTHDLSPGHNYIIGSHPHGILCVGAFCNFITGSTGFSELFPGIRPFLTTLAGNFRLPLFREYLMSGGLCPVTRRAIGHLLSKNGTGNAVAIVIGGAAESLSCSPGVTTLILKNRKGFVRMALQHGAFLVPSFSFGENELFRQVVFEEGSWMRAVQQRFQKMMGFAPCVFYGRGLTSVRSRGFLPYARPITTVVGEPVTVPKIEEPSSEVVDLYHGMYVRSLLKLFNDNKTKASSWRQKSTWPRS

SEQ ID NO: 14 - protein, 352 aa, Simmondsia chinensis
  wax synthase
MEVEKELKTFSEVWISAIAAACYCRFVPAVAPHGGALRLLLLLPVVLLFIFLPLRLSSFHLGGPTALYLVWLANFKLLLFAFHLGPLSNPSLSLLHFISTTLLPIKFRDDPSNDHEKNKRTLSFEWRKVVLFVAKLVFFAGILKIYEFRKDLPHFVISVLYCFHFYLGTEITLAASAVIARATLGLDLYPQFNEPYLATSLQDFWGRRWNLMVSDILGLTTYQPVRRVLSRWVRLRWEVAGAMLVAFTVSGLMHEVFFFYLTRARPSWEVTGFFVLHGVCTAVEMVVKKAVSGKVRLRREVSGALTVGFVMVTGGWLFLPQLVRHGVDLKTIDEYPVMFNYTQKKLMGLLGW

SEQ ID NO: 15 - protein, 608 aa, Arabidopsis thaliana
  wax synthase
MREFVGDGGGPPRWFSPLECGAQATNSPLLLYLPGIDGTGLGLIRHHKKLGEIFDIWCLHIPVSDRTPVKDLVKLIEETVKSENFRLPNRPIYLVGESIGACLALDVAARNPNIDLSLILVNPATHVNNFMVQPLSGMLNVLPDGLPTLLEDIFDFGFKQGDPLTGMLDALSNEFSVQRMGGVGGGMLRDVLAVSANLPTLSRMFPKDTLLWKLEMLKYAIASVNSHIYSVRAETLILLSGRDHWLLKEEDIDRYSRTLPKCIVRKLDDNGQFPLLEDGVDLATIIKCTCFYRRGKSHDHITDYIMPTTFELKQQVDDHRLLMDGTSPVMLSTLEDGTVVRSLEGLPSEGPVLYVGYHMILGFELAPMVIQLMTERNIHLRGLAHPMLFKNLQDSLVDTKMFDKYKIMGGVPVSHFNIYKLLREKAHVLLYPGGVREALHRKGEEYKLFWPERSEFVRVASKFGAKIVPFGVVGEDDICEIVLDSNDQRNIPILKDLMEKATKDAGNIREGDESELGNQECYFPGLVPKIPGRFYYYFGKPIETAGKEKELKDKEKAQELYLQVKSEVEQCIDYLKVKRESDPYRHLLPRMLYQASHGWSSEIPTFDL

(19) United States
(12) Patent Application Publication
(10) Pub. No.: US 2022/0017915 A1
(43) Pub. Date: Jan. 20, 2022

(54) Production of Insect Pheromone Precursors in Plants

(71) Applicants: Per Hofvander, Bjarred (SE); Sten Stymne, Landskrona (SE); Christer Lofstedt, Lund (SE); Bao-Jian Ding, Malmo (SE)

(72) Inventors: Per Hofvander, Bjarred (SE); Sten Stymne, Landskrona (SE); Christer Lofstedt, Lund (SE); Bao-Jian Ding, Malmo (SE)

(21) Appl. No.: 17/498,648

(22) Filed: Oct. 11, 2021

Related U.S. Application Data
(62) Division of application No. 15/764,808, filed on Mar. 29, 2018, now Pat. No. 11,162,111.

(51) Int. Cl. C12N 15/82; C07C 57/03; C07C 69/533; C07C 69/58

(52) U.S. Cl. CPC C12N 15/8286; C12N 15/8247; C07C 69/58; C07C 69/533; C07C 57/03

(57) ABSTRACT

Wax esters (WE) and triacylglycerols (TAG) can be accumulated in large amounts in plant cells. The present invention relates to insect pheromone precursors which are produced as components of WE or TAG in plants. Further the invention concerns means and methods for the production of plant cells producing WE and/or TAG containing insect pheromone precursors in form of either a fatty acid or a fatty alcohol. Said method uses plants where wax esters are modified by desaturase, fatty acyl reductase and wax ester synthase or the triacylglycerol is modified by a desaturase. Finally the insect pheromone precursors can be extracted and used in the manufacturing of insect pheromones from WE and TAG.

### FIELD OF INVENTION

Wax esters (WE) and triacylglycerols (TAG) can be accumulated in large amounts in plant cells. The present invention relates to insect pheromone precursors which are produced as components of WE or TAG in plants. Further the invention concerns means and methods for the production of plant cells producing WE and/or TAG containing insect pheromone precursors in form of either a fatty acid or a fatty alcohol. Finally the invention relates to the extraction of insect pheromone precursors and use of the insect pheromone precursors in the manufacturing of insect pheromones from WE and TAG.

### BACKGROUND OF INVENTION

Pheromones are environmentally friendly alternatives to the use of traditional pesticides for control of insect pests and indeed synthetic pheromones are annually produced in huge amounts for this purpose. The use of pheromones for control of pest insects has many advantages over the use of traditional pesticides. The pheromones are non-toxic; they have no adverse effects on non-target organisms, they do not kill parasitoids or other beneficial insects, and the risks of resistance being developed in the pests are small. Even in terms of profit and reduction in damage pheromones often compare favorably to the use of insecticides. In the case of treating cabbage against diamondback moth infestation, pheromone-based integrated pest management was both cheaper ($62 relative to $123 per ha) and the gross profit was higher (ca $800 compared to $456 per ha) than in ordinary practice with insecticides [1]. The global market for pheromone-based control products is currently estimated to approximately $200 million.

Although prices have come down significantly for synthetic made pheromones commonly used, most prices remain high. Labor costs for the application of pheromones may be high and longevity of the formulations is a concern. Standard approaches to pheromone synthesis either require the use of hazardous chemicals or may result in the production of hazardous waste by-products [2, 3]. The inherent waste problem in synthetic pheromone production could be avoided by inventing and developing an innovative green chemistry alternative, i.e, the synthesis of pheromones or pheromone precursors in a cost-effective and environmentally friendly plant factory. Our strategy involves the use of a cost effective plant factory expressing a suite of biosynthetic enzymes for production of moth pheromones.

Female moths emit species-specific pheromone blends that attract males of the same species over long distances [4]. A majority of the identified moth pheromone compounds consist of fatty acid derivatives produced de novo in the pheromone gland [5, 6]. Their biosynthesis typically involves desaturation of fatty acids, reduction to primary alcohols and further modification to produce acetates or aldehydes.

Great advances have been made during the last 15 years with respect to our understanding of the molecular basis of moth pheromone biosynthesis [7, 8]. Heterologous expression systems have allowed the confirmation of the function of many desaturases [9-13] and fatty-acyl reductases (FARs) [14-16]. Desaturases introduce double bonds in different positions in fatty acids with different chain-length. The functionally characterized FARs converts fatty acyl moieties into fatty alcohols.

Transgenic plants have also proven useful to express enzymes from insects. For example, transgenic plants produced a pheromone precursor upon introduction of a moth desaturase [17] and an aphid alarm pheromone has been produced from endogenous plant sesquiterpene by expression of an (E)-β-farnesene synthase cDNA [18]. Transformation of plants is performed by standard practices and regulation of integrated genes can direct their expression to tissues of choice such as seeds or leaf.

### SUMMARY OF THE INVENTION

The present invention demonstrates for the first time the feasibility of the production and accumulation of large amounts of insect (moth) pheromone precursors in transgenic plants, such as in the leaf cells and oil seed cells as components of WE and/or TAG by the concerted expression of a suite of biosynthetic enzymes.

In a first aspect the invention relates to an isolated insect pheromone precursor or a mixture of isolated insect pheromone precursors produced in plant cells (in vivo) as part of plant wax esters and/or plant triacylglycerols,
a. wherein the wax ester in the plant is at least modified by a desaturase, fatty acyl reductase and a wax ester synthase during synthesis of the pheromone precursor and/or
b. wherein the plant triacylglycerol in the plant is at least modified by a desaturase during synthesis of the pheromone precursor.

In a second aspect the invention relates to a genetically modified plant having incorporated into the genome one or more heterologous gene(s) encoding at least a desaturase, fatty acyl reductase and a wax ester synthase or a desaturase, wherein the plant produces insect pheromone precursors.

In a third aspect the invention relates to an isolated insect pheromone precursor from the transgenic plant disclosed above.

In a final aspect the invention relates to the use of the isolated insect pheromone precursor or the plants according to any of preceding claims for the production of insect pheromones.

The present invention further relates to the extraction of WE and TAG containing insect pheromone precursors from said plant tissues. Methods are described, that are known to persons skilled in the art of organic chemistry, to purify and convert the insect pheromone precursors in WE and TAG into insect pheromone molecules/particles.

By the invention it is for the first time possible to produce insect pheromone precursors environmentally friendly, in large amounts limiting the used chemical reactions to cheap and more benign chemistry.

### BRIEF DESCRIPTION OF THE FIGURES

FIG. 1 shows the synthesis of the insect pheromone precursors and the pathway of their incorporation into either wax ester (WE) or triacylglycerol (TAG). An optional step is the enhanced supply of suitable substrates of 12-16 carbon compounds by the release through a plastid localized acyl-ACP thioesterase, exemplified by CpFATB2. Acyl-CoA substrates are then utilized by desaturases introducing desaturation in specific positions on the acyl chain and with steric configurations that are found in insect pheromone components. The desaturated acyl-CoAs are then either utilized for TAG synthesis or in the presence of a wax synthase incorporated into a wax ester. To form a wax ester, an alcohol component is also needed which is provided by a fatty acyl reductase action on the formed desaturated acyl-CoA. In this aspect both the alcohol as well as the fatty acid part of the wax ester can subsequently be utilized for insect pheromone component production.

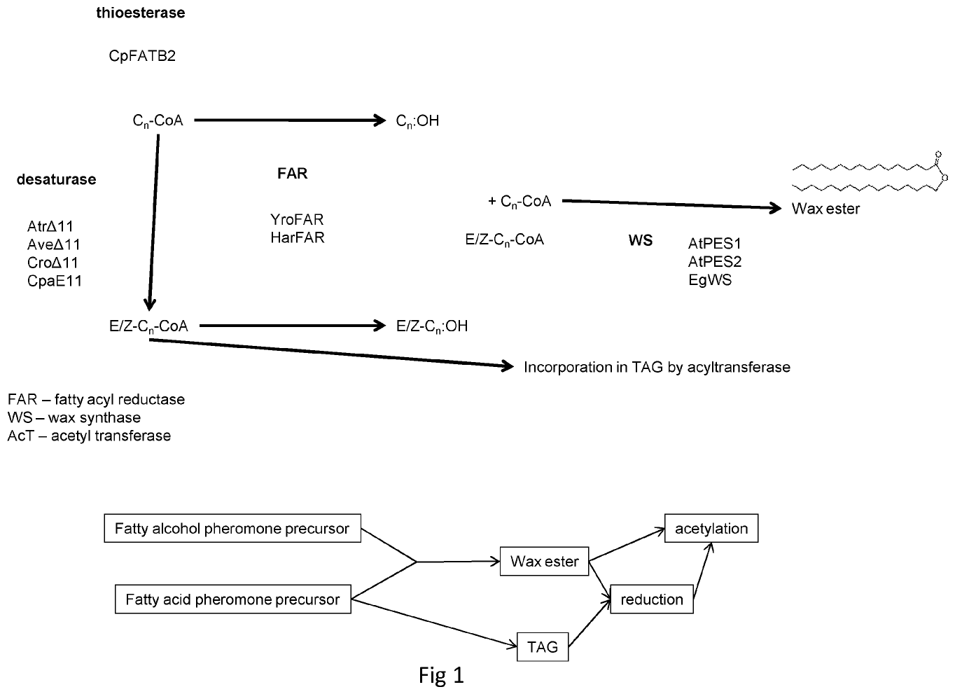

### DETAILED DESCRIPTION OF THE INVENTION

### Definitions

In the context of the present application and invention, the following definitions apply:

The term “genetic modification” means the introduction of homologous and/or heterologous foreign nucleic acid molecules into the genome of a plant cell or into the genome of a plant, wherein said introduction of these molecules leads to an accumulation of insect pheromone precursors as constituents of WE and/or TAG in seed or vegetative tissue of a plant.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

Genes and Enzymes for the Production of Insect Pheromone Precursors and Introduction into WE and TAG in Plant Cells.

In one embodiment the invention relates to an isolated insect pheromone precursor or a mixture of isolated insect pheromone precursors produced in plant cells (in vivo) as part of plant wax esters and/or plant triacylglycerols,
a. wherein the wax ester in the plant is at least modified by a desaturase, fatty acyl reductase and a wax ester synthase during synthesis of the pheromone precursor and/or
b. wherein the plant triacylglycerol in the plant is at least modified by a desaturase during synthesis of the pheromone precursor.

The produced plant wax esters and/or plant triacylglycerols may then be used for the production of insect pheromones and further used for pest management. By the invention the plant wax esters and/or plant triacylglycerols can be produced by an environmental friendly process as well as in an economically beneficial way. The amount of isolated insect pheromone precursor or a mixture of isolated insect pheromone precursors may further be enhanced by a plastidic acyl-ACP thioesterase providing an increased amount of suitable substrates in the plant either prior or after other modifications. However, the modifications occur in vivo in the plant by enzymes expressed by the plant prior to that the insect pheromone precursors are to be isolated. The genes that are to produce the insect pheromone precursors are heterologous genes and express at least the following enzymes acyl-CoA desaturase, fatty acyl reductase, wax ester synthase and/or plastidic acyl-ACP thioesterase, which are involved in the provision of substrates or synthesis of the wax esters and/or triacylglycerols. Examples includes precursors selected from the group consisting of unsaturated fatty acids (E)-11-tetradecanoyl (E11-14), (Z)-11-tetradecanoyl (Z11-14) and (Z)-11-hexadecanoyl (Z11-16) and unsaturated alcohols (E)-11-tetradecenol (E11-14: OH), (Z)-11-tetradecenol (Z11-14:OH) and (Z)-11-hexadecenol (Z11-16:OH) or a mixture thereof. The isolated insect pheromone precursor or a mixture thereof, may be produced in the seeds or vegetative tissue of the plant.

In one embodiment, the invention relates to a genetically modified plant having incorporated into the genome a heterologous gene encoding at least an acyl-CoA desaturase, fatty acyl reductase and a wax ester synthase or an acyl-CoA desaturase, wherein the plant produced insect pheromone precursors incorporated as part of wax ester and/or TAG. The genetically modified plant may further have incorporated in the genome a heterologous gene encoding at least a plastidic acyl-ACP thioesterase, such as having at least a desaturase, fatty acyl reductase, a plastidic acyl-ACP thioesterase and a wax ester synthase or a desaturase and a plastidic acyl-ACP thioesterase.

In one example the acyl-CoA pool for moth pheromone precursors is optimized in the plant cells by the introduction of genes encoding plastidic acyl-ACP thioesterases that release acyl chains of a desired length for moth pheromone precursor production. Moth pheromone precursors are first produced as monounsaturated acyl-CoA and monounsaturated fatty alcohol. Insect genes encoding desaturases are used to firstly produce desired acyl-CoA dependent on the specific desaturase and insect genes encoding fatty acyl reductases (FAR) are used to secondly reduce monounsaturated acyl-CoA into their corresponding alcohols.

Insect pheromone precursors produced by desaturases and in the case of wax esters, also by FAR, are then used as substrates for the assembly of TAG and WE. To introduce insect pheromone precursors into WE, genes encoding the function of wax ester synthase are used. Examples of genes utilized encoding a function of wax ester synthase are, but not limited to, Arabidopsis thaliana phytol ester synthase 1 (AtPES1), Arabidopsis thaliana phytol ester synthase 2 (AtPES2) or Euglena gracilis wax ester synthase (EgWS) [19, 20]. As native enzymes, AtPES1 and AtPES2 are imported into plastid organelles. For use in the present invention DNA sequences coding for a signal transit peptide may be deleted from AtPES1 and AtPES2 and replaced with an ATG codon for new translation start. Enzymes are preferentially targeted to the endoplasmic reticulum (ER). Means for attaching ER localization or retainment signals are well known in the art. Monounsaturated acyl-CoA insect precursors as well as monounsaturated fatty alcohol insect pheromone precursors are introduced into the WE. For TAG produced monounsaturated acyl-CoA insect pheromone precursors can be accepted by available enzymes in the plant system for TAG assembly, although the introduction of genes encoding enzymes preferentially utilizing the medium chain monounsaturated acyl-CoA insect pheromone precursors may be preferred. Examples of genes used to enhance the introduction of monounsaturated acyl-CoA insect pheromone precursors into TAG are, but not limited, to glycerol-3-phosphate acyl transferase, lysophosphatidic acid acyltransferase and diacylglycerols acyltransferase encoding genes. In order to enhance the production of TAG and WE, transcription factors enhancing plant cell fatty acid synthesis such as, but not limited to, WRINKLED1 type of transcription factors [21] can be co-expressed. To prevent degradation of WE and TAG produced in leaf cells, oleosin genes can be co-expressed.

The genetically modified plant disclosed above may comprise at least one gene that encodes a polypeptide, wherein the polypeptide is/are selected from the group consisting of SEQ ID NO: 1, 2, 3, 4, 5, 6, 7, 8, 9, 10, 11, 12, 13, 14 or 15 or a polypeptide having 60% identity to a polypeptide with an amino acid sequence of SEQ ID NO: 1, 2, 3, 4, 5, 6, 7, 8, 9, 10, 11, 12, 13, 14 or 15. The term “identity” means a sequence identity over the whole length of the coding region of at least 60%, such as 65, 70, 75, 80, 85, 90, 91, 92, 93, 94, 95, 96, 97, 98 or 99%.

The term “identity” is to be understood to mean the number of amino acids/nucleotides (identity) corresponding with other proteins/nucleic acids, expressed as a percentage. Identity is preferably determined by comparing SEQ ID NO: 1, 2, 3, 4, 5, 6, 7, 8, 9, 10, 11, 12, 13, 14 or 15 for amino acids with other proteins acids with the help of computer programs. If sequences that are compared with one another have different lengths, the identity is to be determined in such a way that the number of amino acids, which have the shorter sequence in common with the longer sequence, determines the percentage quotient of the identity. Preferably, identity is determined by means of the computer program ClustalW, which is well known and available to the public (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from different Internet sites, including the IGBMC (Institut de Genetique et de Biologie Moleculaire et Cellulaire, B.P.163, 67404 Illkirch Cedex, France; ftp://ftp-igbmc.u-strasbg.fr/pub/) and the EBI (ftp://ftp.ebi.ac.uk/pub/software/) as well as from all mirrored Internet sites of the EBI (European Bioinformatics Institute, Wellcome Trust Genome Campus, Hinxton, Cambridge CB10 1SD, UK).

In one embodiment the genetically modified plant comprises at least one, two, three or four genes that encodes a polypeptide, wherein the polypeptide is/are selected from the group consisting of SEQ ID NO: 1, 2, 3, 4, 5, 6, 7, 8, 9, 10, 11, 12, 13, 14 or 15 or a polypeptide having 60% identity to a polypeptide with an amino acid sequence of SEQ ID NO: 1, 2, 3, 4, 5, 6, 7, 8, 9, 10, 11, 12, 13, 14 or 15. One example being a genetically modified plant which comprises one, two, three or four polypeptides selected from the group consisting of SEQ ID NO: 1 and 2, 3, 4, 5 or 6 and 7, 8, 9 or 10 and 11, 12, 13, 14 or 15.

The genetically modified plant may be any plant species capable of accumulating oil in the form of WE and/or TAG in vegetative or seed tissue. For seed tissue, in particular oil seed crops as Camelina sativa, Crambe abyssinica Brassica species are applicable while for vegetative tissue species capable of producing large amounts of biomass such as tobacco, miscanthus or sugar beet. However, depending on where the plants are to be produced other tissues may be the choice as well as other species.

The genetically modified plant described above may express/produce the wax esters and/or triacylglycerol in the seeds or in the vegetative tissue or in any other useful tissue. In one example the wax esters and/or triacylglycerol are produced in the seeds.

In another embodiment the invention relates to isolated insect pheromone precursor, wherein the isolated insect pheromone precursors are isolated from a transgenic plant, such as the transgenic plant (genetically modified plant) described above.

Expression Vectors for the Introduction of Genes into a Heterologous Production System

By expression vector is meant a vector suitable for the stable introduction of genes into an organism of choice and their subsequent controlled expression in a desirable context. For expression of a gene encoding an enzymatic function a promoter is commonly attached to the front end of the gene while a terminator is attached to the back end of the gene. The promoter defines in which tissue, under which conditions and to which extend a gene is expressed. The terminator defines the end of a gene and terminating transcription. Promoters and terminators are well known in the art. Examples of promoters are for seed expression the napin or glycinin promoter and for vegetative tissue the CaMV35S promoter but other promoters known in the art can be used to practice the invention.

Other examples of promoters are, for example, the ubiquitin promoter from maize for constitutive expression, the patatin promoter B33 for tuber-specific expression in potatoes, the USP promoter, the phaseolin promoter, promoters of zein genes from maize, glutelin promoter or shrunken-1 promoter.

Furthermore, a termination sequence (polyadenylation signal) may be present, which is used for adding a poly-A tail to the transcript. A function in the stabilisation of the transcripts is ascribed to the poly-A tail. Elements of this type are described in the literature and can be exchanged at will.

An expression vector may contain one or several of the genes which are to be introduced. Genes on an expression vector to be introduced for the production of insect pheromone precursors as part of WE and TAG may be expressed from the same or by different promoters.

Methods for the creation of expression vectors according to the invention are known to the person skilled in the art, and include genetic methods such as bonding nucleic acid molecules by way of ligation, genetic recombination, or new synthesis of nucleic acid molecules, for example (see e.g. Sambrok et al., Molecular Cloning, A Laboratory Manual, 3rd edition (2001) Cold Spring Harbour Laboratory Press, Cold Spring Harbour, N.Y. ISBN: 0879695773, Ausubel et al., Short Protocols in Molecular Biology, John Wiley & Sons; 5th edition (2002), ISBN: 0471250929).

### Transformation and Regeneration

By transformation is meant the stable introduction of expression vectors enabling the production of insect pheromone precursors and their introduction into WE and TAG. The transformation of plants is standard technology and well known in the art. Commonly this is performed by Agrobacterium tumefaciens transformation of plant tissues. Well known in the art is that different tissues of different species are differently accessible for transformation. Plant transformation is as well known in the art not limited to Agrobacterium tumefaciens but genes can also be introduced by other means well-known for a person skilled in the art. To select for which cells have stably introduced desired genes, commonly a selective marker is contained on the expression vector used for transformation. Examples of selection agents among others are kanamycin, hygromycin and BASTA but as well known in the art different selection systems can be used for successful selection of cells which have stably integrated genes for expression. Methods for the regeneration of shoots from selected transgenic cells are well known in the art. Regenerated plants can be analyzed for their transgenic status by PCR or Southern blotting and the expression of genes can be analyzed by PCR or Northern blotting. Translation into corresponding proteins can be analyzed by Western.

### Cultivation of Plant Material for Extraction of WE and TAG Containing Insect Pheromone Precursors

The transgenic plants can be grown either in climate chambers, in greenhouse or in field. Harvesting of leaf or seed material for WE and TAG containing insect pheromone precursors is done at the optimal stage of accumulation per area cultivated. For production of pheromone precursors in leaves, any transformable broad leaf plant is preferentially used. For production of pheromone precursors in seeds any transformable oil seed producing plant can be used. Nicotiana tabaccum could be used for transformation of genes for insect precursor production as components of WE and/or TAG in vegetative tissue while Camelina sativa is an example of a plant species for transformation of genes for insect precursor production as components of WE and/or TAG in oil seeds.

Producing Insect Pheromones from Harvested Material

All chemical steps described below are familiar to persons skilled in the art of organic chemistry.

1) Extraction of leaves or seed, depending on the tissue specificities of the promoters used to govern the expression of the introduced genes. The extraction of the tissue with organic solvents such as n-heptane and n-hexane will yield fractions with TAG and WE in high purity.

2) The production of active pheromone from oil with wax esters containing precursor pheromone fatty alcohol can be done by fractionation and chemical conversion procedures known to the person skilled in the art by following methods, but not excluding other known methods. The wax esters can be separated from triacylglycerol molecules by e.g. short path distillation, by crystallization, by silica gel column chromatography or by mild saponification of the triacylglycerols in the oil by sodium or potassium hydroxide in water solution at room temperature and extracting the intact wax esters with organic solvent, i.e. hexane. The wax esters are hydrolyzed to fatty acid and fatty alcohols by treatment with sodium or potassium hydroxide in water solution at elevated temperature. The fatty alcohols are extracted into organic solvent such as heptane and acetylated with treatment with acetylchloride or acetic acid anhydride to yield the final active pheromone. In case of aldehyde pheromone molecules, the alcohol can be partially re-oxidized to yield a fatty aldehyde. Acetylated fatty alcohols and fatty aldehydes can easily be separated from the fatty acids by e.g. distillation. The TAG fraction can easily be hydrolyzed by alkali to yield free fatty acids. The pheromone precursor component fatty acid can be separated from other fatty acids by different means such as distillation or by reverse phase chromatography. The carboxylic group of the fatty acid can then be selectively reduced without reducing the double bonds to yield the alcohol component of the pheromone. The fatty alcohol can either be acetylated in case of acetylated pheromones as described above or partially oxidized to aldehyde to yield an aldehyde pheromone.

In a final embodiment the invention relates to the use of the isolated insect pheromone precursor or the plants according to any of preceding claims for the production of pheromones.

### EXAMPLES

### Example 1

Production of insect pheromone precursors as constituents of wax esters (WE) For precursor production as constituents of wax ester in seed oil, genes coding for Cuphea palustris plastidic acyl-ACP thioesterase (CpFATB2), Argyrotaenia velutinana desaturase (AveΔ11), Amyelois transitella desaturase (AtrΔ11), Helicoverpa armigera fatty acyl reductase (HarFAR) and the mature form of Arabidopsis thaliana phytol ester synthase 2 lacking plastid transit peptide sequence (mAtPES2) were respectively cloned between a glycinin or napin promoter and a glycinin or nopaline synthase terminator. The genes fused to glycinin or napin promoter and glycinin or nopaline synthase terminator were inserted between T-DNA right and left border sequences of a binary vector where also an nptII gene for selection of transgenic cells was situated. Camelina sativa plants were transformed using floral dipping and produced seeds germinated on kanamycin containing medium. Transformed plants were selfed and seeds collected upon maturation. Lipids were extracted from seeds and then separated using thin layer chromatography (TLC). The WE fraction was identified using a WE standard and samples were scraped from the TLC plate. The wax ester samples were hydrolyzed and methyl esters (ME) and fatty alcohols were separated and identified using GC/MS yielding (E)-11-tetradecanoyl-ME (E11-14-ME), (Z)-11-tetradecanoyl-ME (Z11-14-ME), (Z)-11-hexadecanoyl-ME (Z11-16-ME) and corresponding alcohols (E)-11-tetradecenol (E11-14:OH), (Z)-11-tetradecenol (Z11-14: OH), (Z)-11-hexadecenol (Z11-16:0H).

For precursor production as constituents of wax ester in vegetative leaf tissue, genes coding for Cuphea palustris plastidic acyl-ACP thioesterase (CpFATB2), Argyrotaenia velutinana desaturase (AveΔ11), Amyelois transitella desaturase (AtrΔ11), Helicoverpa armigera fatty acyl reductase (HarFAR), Arabidopsis thaliana WRINKLED 1 (AtWRI1) and the mature form of Arabidopsis thaliana phytol ester synthase 2 lacking plastid transit peptide sequence (mAtPES2) were respectively cloned between a CaMV35S promoter and a nopaline synthase terminator. The genes fused to CaMV35S promoter and nopaline synthase terminator were inserted between T-DNA right and left border sequences of a binary vector where also an nptII gene for selection of transgenic cells was situated. Nicotiana tabaccum leaf discs were transformed and shoots regenerated on kanamycin containing medium. Shoots were transferred to soil and allowed to develop. Lipids were extracted from expanded leaves and then separated using thin layer chromatography (TLC). The WE fraction was identified using a WE standard and samples were scraped from the TLC plate. The wax ester samples were hydrolyzed and methyl esters (ME) and fatty alcohols were separated and identified using GC/MS yielding (E)-11-tetradecanoyl-ME (E11-14-ME), (Z)-11-tetradecanoyl-ME (Z11-14-ME), (Z)-11-hexadecanoyl-ME (Z11-16-ME) and corresponding alcohols (E)-11-tetradecenol (E11-14:OH), (Z)-11-tetradecenol (Z11-14:OH), (Z)-11-hexadecenol (Z11-16: OH). Tobacco leaves infiltrated with Agrobacterium containing different gene constructs in the following two combinations, CpaFATB2+HarFAR+AveΔ11+mAtPES2+AtWRI1, CpaFATB2+HarFAR+AtrΔ11+mAtPES2+AtWRI1 Yielded 0.75 and 0.91 μg 14:1 and 0 and 2.83 μg 16:1 leaf fresh weight respectively as insect pheromone precursor constituents of isolated wax ester.

### Example 2

### Production of Desaturated Insect Pheromone Precursors as Constituent of Triacylglycerols (TAG).

For precursor production as constituents of triacylglycerol (TAG) in seed oil, genes coding for Cuphea palustris plastidic acyl-ACP thioesterase (CpFATB2), Choristoneura parallela desaturase (CpaE11), Argyrotaenia velutinana desaturase (AveΔ11), Amyelois transitella desaturase (AtrΔ11) were respectively cloned between a glycinin or napin promoter and a glycinin or nopaline synthase terminator. The genes fused to glycinin or napin promoter and glycinin or nopaline synthase terminator were inserted between T-DNA right and left border sequences of a binary vector where also an nptII gene for selection of transgenic cells was situated. Camelina sativa plants were transformed using floral dipping and produced seeds germinated on kanamycin containing medium. Transformed plants were selfed and seeds collected upon maturation. Lipids were extracted from seeds and then separated using thin layer chromatography (TLC). The TAG fraction was identified using a TAG standard and samples were scraped from the TLC plate. The TAG samples were hydrolyzed and methyl esters (ME) were separated and identified using GC/MS yielding (E)-11-tetradecanoyl-ME (E11-14-ME), (Z)-11-tetradecanoyl-ME (Z11-14-ME), (Z)-11-hexadecanoyl-ME (Z11-16-ME).

Camelina sativa (high myristic) transformed with CpaE11 yielded isolated seed TAG containing in individual seeds 7.1, 7.2, 7.6, 7.6, 8.0, 8.2 and 9.2% E11-14 of total fatty acids. Camelina sativa (high myristic) transformed with AveΔ11 yielded isolated seed TAG containing in individual seeds 2.0, 2.1, 2.2 and 2.3% E11-14 and 6.0, 6.1, 6.5 and 6.8% Z11-14 of total fatty acids. Camelina sativa (high palmitic) transformed with AtrΔ11 yielded isolated seed TAG containing in individual seeds 17.3, 17.4, 17.7, 19.2 and 28.2% Z11-16 of total fatty acids. For precursor production as constituents of TAG in vegetative leaf tissue, genes coding for Cuphea palustris plastidic acyl-ACP thioesterase (CpFATB2), Argyrotaenia velutinana desaturase (AveΔ11), Amyelois transitella desaturase (AtrΔ11) and Arabidopsis thaliana WRINKLED 1 (AtWRI1) were respectively cloned between a CaMV35S promoter and a nopaline synthase terminator. The genes fused to CaMV35S promoter and nopaline synthase terminator were inserted between T-DNA right and left border sequences of a binary vector where also an nptII gene for selection of transgenic cells was situated. Nicotiana tabaccum leaf discs were transformed and shoots regenerated on kanamycin containing medium. Shoots were transferred to soil and allowed to develop. Lipids were extracted from expanded leaves and then separated using thin layer chromatography (TLC). The TAG fraction was identified using a TAG standard and samples were scraped from the TLC plate. The TAG samples were hydrolyzed and methyl esters (ME) were separated and identified using GC/MS yielding (E)-11-tetradecanoyl-ME (E11-14-ME), (Z)-11-tetradecanoyl-ME (Z11-14-ME), (Z)-11-hexadecanoyl-ME (Z11-16-ME).

### Example 3

The production of active pheromone from TAG (triacylglycerols) containing precursor pheromone fatty acids was performed by fractionation and chemical conversion procedures. The oil was treated with water free methanol containing acid or alkali as catalysts to yield fatty acid methyl esters. The fatty acid of interest was purified as methyl esters by short path distillation or by crystallization and the fatty acid was converted to corresponding fatty acid alcohol by reaction with lithiumaluminium hydride or tri-tert-butoxyaluminumlithium hydride or by selective catalytic hydrogenation by high pressure hydrogenation. Resulting fatty alcohol was acetylated by treatment with acetylchloride or acetic acid anhydride to yield the final active pheromone molecule.

### Example 4

The production of active pheromone from oil with wax esters containing precursor pheromone fatty alcohol was performed by fractionation and chemical conversion procedures. The wax esters were separated from triacylglycerol molecules by short path distillation, by crystallization, by silica gel column chromatography or by mild saponification of the triacylglycerols in the oil by sodium or potassium hydroxide in water solution at room temperature and extracting the intact wax esters with hexane. The wax esters were hydrolyzed to fatty acid and fatty alcohols by treatment with sodium or potassium hydroxide in water solution at elevated temperature. The fatty alcohols were extracted into heptane and acetylated with treatment with acetylchloride or acetic acid anhydride to yield the final active pheromone.

### REFERENCES

1. Reddy, G. V. P. and A. Guerrero, Pheromone-based integrated pest management to control the diamondback moth Plutella xylostella in cabbage fields. Pest Management Science, 2000. 56(10): p. 882-888.
2. Mori, K., The Synthesis of Insect Pheromones, 1979-1989, in Total Synthesis of Natural Products 2007, John Wiley & Sons, Inc. p. 1-521.
3. Mori, K., Chemical Synthesis of Hormones, Pheromones and Other Bioregulators 2010: John Wiley & Sons Ltd.
4. Wyatt, T. D., Pheromones and animal behaviour: communication by smell and taste. Pheromones and animal behaviour: communication by smell and taste. 2003. i-xv, 1-391.
5. Ando, T., S. Inomata, and M. Yamamoto, Lepidopteran sex pheromones. Chemistry of Pheromones and Other Semiochemicals I, 2004. 239: p. 51-96.
6. Tillman, J. A., et al., Insect pheromones—an overview of biosynthesis and endocrine regulation. Insect Biochemistry and Molecular Biology, 1999. 29(6): p. 481-514.
7. Knipple, D. C. and W. L. Roelofs, Molecular biological investigations of pheromone desaturases. Insect pheromone biochemistry and molecular biology: the biosynthesis and detection of pheromone and plant volatiles., ed. G. J. Blomquist and R. G. Vogt 2003. 81-106.
8. Lassance, J.-M., et al., Functional consequences of sequence variation in the pheromone biosynthetic gene pgFAR for Ostrinia moths. Proceedings of the National Academy of Sciences of the United States of America, 2013. 110(10): p. 3967-3972.
9. Hao, G., et al., Characterization of Z/E11- and Z9-desaturases from the obliquebanded leafroller moth, Choristoneura rosaceana. Journal of insect science (Online), 2002. 2: p. 26-26.
10. Lienard, M. A., et al., Key biosynthetic gene subfamily recruited for pheromone production prior to the extensive radiation of Lepidoptera. Bmc Evolutionary Biology, 2008. 8.
11. Liu, W. T., et al., Moth desaturase characterized that produces both Z and E isomers of Delta 11-tetradecenoic acids. Insect Biochemistry and Molecular Biology, 2002. 32(11): p. 1489-1495.
12. Liu, W. T., et al., Desaturases from the spotted fireworm moth (Choristoneura parallela) shed light on the evolutionary origins of novel moth sex pheromone desaturases. Gene, 2004. 342(2): p. 303-311.
13. Roelofs, W. L., et al., Evolution of moth sex pheromones via ancestral genes. Proceedings of the National Academy of Sciences of the United States of America, 2002. 99(21): p. 13621-13626.
14. Lassance, J.-M., et al., Allelic variation in a fatty-acyl reductase gene causes divergence in moth sex pheromones. Nature, 2010. 466(7305): p. 486-U7.
15. Lienard, M. A., et al., Evolution of multicomponent pheromone signals in small ermine moths involves a single fatty-acyl reductase gene. Proceedings of the National Academy of Sciences of the United States of America, 2010. 107(24): p. 10955-10960.
16. Moto, K. i., et al., Pheromone gland-specific fatty-acyl reductase of the silkmoth, Bombyx mori. Proceedings of the National Academy of Sciences, 2003. 100(16): p. 9156-9161.
17. Nesnerova, P., et al., First semi-synthetic preparation of sex pheromones. Green Chemistry, 2004. 6(7): p. 305-307.
18. Beale, M. H., et al., Aphid alarm pheromone produced by transgenic plants affects aphid and parasitoid behavior. Proceedings of the National Academy of Sciences of the United States of America, 2006. 103(27): p. 10509-10513.
19. Lippold, F., et al., Fatty Acid Phytyl Ester Synthesis in Chloroplasts of Arabidopsis. Plant Cell, 2012. 24(5): p. 2001-2014.
20. Teerawanichpan, P. and X. Qiu, Fatty Acyl-CoA Reductase and Wax Synthase from Euglena gracilis in the Biosynthesis of Medium-Chain Wax Esters. Lipids, 2010. 45(3): p. 263-273.
21. Cernac, A. and C. Benning, WRINKLED1 encodes an AP2/EREB domain protein involved in the control of storage compound biosynthesis in Arabidopsis. Plant Journal, 2004. 40(4): p. 575-585.

1. (canceled)

2. (canceled)

3. (canceled)

4. (canceled)

5. (canceled)

6. A genetically modified plant having incorporated into the genome a heterologous gene encoding at least a desaturase, fatty acyl reductase and/or a wax ester synthase or a desaturase, wherein the plant produces insect pheromone precursors.

7. The genetically modified plant according to claim 6, having incorporated into the genome a further heterologous gene encoding at least a plastidic acyl-ACP thioesterase.

8. The genetically modified plant according to claim 6, wherein the genes encodes a polypeptide selected from the group consisting of SEQ ID NO: 1, 2, 3, 4, 5, 6, 7, 8, 9, 10, 11, 12, 13, 14 or 15 or a polypeptide having 60% identity to a polypeptide with an amino acid sequence of SEQ ID NO: 1, 2, 3, 4, 5, 6, 7, 8, 9, 10, 11, 12, 13, 14 or 15.

9. The genetically modified plant according to claim 8, wherein the genes encodes a polypeptide having 65, 70, 75, 80, 85, 90, 91, 92, 93, 94, 95, 96, 97, 98, or 99% identity to a polypeptide with an amino acid sequence of SEQ ID NO: 1, 2, 3, 4, 5, 6, 7, 8, 9, 10, 11, 12, 13, 14 or 15.

10. The genetically modified plant according to claim 6, wherein the plant species is recognized as an oilseed crop or large biomass producer.

11. The genetically modified plant according to claim 6, wherein the wax esters and/or triacylglycerol is produced in the seeds or in the vegetative tissue.

12. The genetically modified plant according to claim 11, wherein the wax esters and/or triacylglycerol are produced in the seeds.

13. Isolated insect pheromone precursor from the transgenic plant according to claim 6.

14. Use of the plant according to claim 6 for the production of pheromones.
